(19) United States
(12) Patent Application Publication
(10) Pub. No.: US 2011/0276082 A1
(43) Pub. Date: Nov. 10, 2011

(54) Device for providing therapeutic pressure therapy to parts of the body for treatment of pain

(76) Inventor: Robert G. Fehring, Islip, NY (US)

(21) Appl. No.: 12/806,309

(22) Filed: Aug. 10, 2010

Related U.S. Application Data
(60) Provisional application No. 61/276,897, filed on Sep. 17, 2009.

(51) Int. Cl. A61B 17/00

(52) U.S. Cl. 606/204

(57) ABSTRACT

A device for providing therapeutic pressure therapy to parts of the body for treatment of pain which comprises a jaw assembly having a front opening to receive a part of the body therein. A set of inflatable pneumatic bladders is secured in said jaw assembly. A mechanism within the enclosure is for inflating and deflating the pneumatic bladders together at predetermined timed intervals to apply varying amounts of pressure to the part of the body to provide relief from pain.

### CROSS REFERENCE TO RELATED APPLICATIONS

The instant application is a nonprovisional application of U.S. provisional application No. 61/276,897, filed on Sep. 17, 2009, and entitled “Squeeze-Reliever” inflatable pressurized bladders in housing pain reliever, and it is respectfully requested that this application be accorded the benefit under 35 USC 119(e) of said U.S. provisional application.

### BACKGROUND OF THE INVENTION

1. Field of the Invention

The present invention relates to a pain relief apparatus, and more particularly, a device for providing therapeutic pressure therapy to parts of the body for treatment of pain.

2. Description of the Prior Art

Numerous innovations for pressure applying devices have been provided in the prior art that will be described. Even though these innovations may be suitable for the specific individual purposes which they address, however, they differ from the present invention.

A FIRST EXAMPLE, U.S. Pat. No. 3,581,740, Issued on Jun. 1, 1971, to Sherbourne teaches an inflatable splint for extending the fingers and wrist of the hand of the wearer. The splint has an inflatable chamber extending over the hand and wrist area of the wearer, with one surface which assumes a planar configuration when the splint is inflated. The one surface has finger loops adapted to receive the fingers of the wearer and positioned such that, upon inflation, the fingers will be moved to a normally extended, spaced-apart position. Wrist straps on the wrist area are provided to encircle the wearer's wrist so that, upon inflation, the wrist joint of the wearer will be moved to a straight position along with the extended fingers.

A SECOND EXAMPLE, U.S. Pat. No. 3,595,225, Issued on Jul. 27, 1971, to Beeman teaches a support or brace means applicable to portions of a human hand and comprising a twisted, substantially U-shaped, resilient body portion terminating in pressure applying end portions. The end portions apply pressure in substantially opposite directions. The supports of the present invention may be applied to the hand so as to apply substantially oppositely directed pressures to the distal and proximal metacarpal joints of the thumb or first digit, and they may be applied to any of the four remaining digits to exert opposite pressures to adjacent digital bones or phalanges to correct misalignment brought about by arthritis and the like, and to relieve the attendant pain.

A THIRD EXAMPLE, U.S. Pat. No. 3,654,917, Issued on Apr. 11, 1972, to Dienner teaches an electro-magnetic device having a plurality of movable applicators adapted to be brought into yielding engagement with an affected finger, and vibrated. The applicators, as a group, surround the finger and are pressed against the sides of the finger and are moved lengthwise of the finger back and forth over one or the other of the two free finger joints with a stroking motion.

A FOURTH EXAMPLE, U.S. Pat. No. 4,182,338, Issued on Jan. 8, 1980, to Stanulis teaches a device which prevents bleeding through needle puncture wounds by applying pressure to the wound through an elastomeric appliance having a blunt skin abutting surface held in place over the wound by securing straps. The pressure applied by the device is sufficient only to prevent bleeding through the wound and does not impede the subsurface flow of blood.

A FIFTH EXAMPLE, U.S. Pat. No. 5,695,520, Issued on Dec. 9, 1997, to Bruckner et al. teaches a pressure-applying device for relieving distress that includes a pressure applicator having a plate member and a pressure-applying body attached to and protruding from one of a pair of opposite surfaces of the plate member. The plate member has a central portion with a pair of opposite surfaces facing in opposite directions and a peripheral portion surrounding the central portion and having a pair of first opposite sides respectively connected to opposite ends of the central portion and a pair of second opposite sides spaced from a pair of opposite edges of the central portion so as to define respective elongated enclosed slots within the plate member between the second opposite sides of the peripheral portion and the pair of opposite edges of the central portion of the plate member. The device also includes a flexible strap attached to the plate member and adapted for wrapping and securing around a part of the body of a user so that the pressure-applying body is between the flexible strap and part of the body and is in contact with a selected point on the part of the body of the user for applying pressure thereto.

A SIXTH EXAMPLE, U.S. Pat. No. 5,788,657, Issued on Aug. 4, 1998, to Burns teaches a small hand-held device having a body of electrically insulating material, and a central rod of electrically conductive material which provides for the rod extending beyond a proximal and a distal ends of the body to form electrical contacts. Therefore an electrical conduction path is set-up between the contacts. This path is advantageously used to establish a charge state on the skin surface so as to help in relieving localized pain. A pointed proximal end of the device enables an effective massaging action when the device is brought into contact with the skin and pressed into it.

A SEVENTH EXAMPLE, U.S. Pat. No. 5,837,004, Issued on Nov. 17, 1998, to Lavore teaches a device to reduce pain, relax muscle spasm, decrease swelling, and increase circulation to the bones, joints, and muscles of the face of a patient having temporomandibular joint dysfunction, the device having a thin, hexagonal configuration with an essentially uniform cross-section, and a flexible outer envelope having a perimeter limited to covering an area on one side of a patient's face to minimally include the area over the rear portion of the patient's mandible, the zygomatic arch, the front portion of the temporal bone, the great wing of the sphenoid bone, the lower front portion of the parietal bone, the outer envelope also having hook material on both sides of the upper panel placed away from the patient's face so that it can also be used inside-out against the opposite side of the patient's face and the lower rear portion of the frontal bone. Also the outer envelope is secured to the treated area by a separate quick-release non-stretchable strap comprising pile material on one of its sides, and the outer envelope has a removable inner member comprising a gel that can be heated and chilled, without hardening. Applications of the present invention, although not limited to the minimizing of pain, muscle spasms, and inflammation of joints associated with temporomandibular joint dysfunction are best suited for such purposes since its perimeter closely approximates the area on a patient's face which contains pain sensitive structures most commonly affected by temporomandibular joint dysfunction.

It is apparent now that numerous innovations for pressure applying devices have been provided in the prior art that are adequate for various purposes. Furthermore, even though these innovations may be suitable for the specific individual purposes which they address, however, they would not be suitable for the purposes of the present invention as heretofore described.

### SUMMARY OF THE INVENTION

AN OBJECT of the present invention is to provide a device for providing therapeutic pressure therapy to parts of the body for treatment of pain that avoids the disadvantages of the prior art.

ANOTHER OBJECT of the present invention is to provide a device for providing therapeutic pressure therapy to parts of the body for treatment of pain that is simple and inexpensive to manufacture.

STILL ANOTHER OBJECT of the present invention is to provide a device for providing therapeutic pressure therapy to parts of the body for treatment of pain that is simple to use.

BRIEFLY STATED, STILL YET ANOTHER OBJECT of the present invention is to provide a device for providing therapeutic pressure therapy to parts of the body for treatment of pain which comprises a housing assembly having a front opening to receive the part of the body therein. A first pneumatic bladder is in the housing assembly at bottom of the front opening. A second pneumatic bladder is in the housing assembly at top of the front opening directly above the first pneumatic bladder. An enclosure is behind the housing assembly. A mechanism within the enclosure is for inflating and deflating the first pneumatic bladder and the second pneumatic bladder together at predetermined timed intervals to apply varying amounts of pressure to the part of the body to provide relief from pain.

The novel features which are considered characteristic of the present invention are set forth in the appended claims. The invention itself, however, both as to its construction and its method of operation, together with additional objects and advantages thereof, will be best understood from the following description of the specific embodiments when read and understood in connection with the accompanying drawings.

### BRIEF DESCRIPTION OF THE DRAWING

The figures of the drawings are briefly described as follows:

FIG. 1 is a diagrammatic top, left, front perspective view of an embodiment of the present invention;

FIG. 2 is a diagrammatic bottom, left, front perspective view thereof;

FIG. 3 is a diagrammatic top, left, rear perspective view thereof;

FIG. 4 is a diagrammatic left side elevational view taken in the direction of arrow 4 in FIG. 1;

FIG. 5 is a diagrammatic top view taken in the direction of arrow 5 in FIG. 1;

FIG. 6 is a diagrammatic front view taken in the direction of arrow 6 in FIG. 1;

FIG. 7 is a diagrammatic top, left, front perspective view of the stationary lower jaw component per se;

FIG. 8 is a diagrammatic top view taken in the direction of arrow 8 in FIG. 7;

FIG. 9 is a diagrammatic front view taken in the direction of arrow 9 in FIG. 7;

FIG. 10 is a diagrammatic top, left, front perspective view of the movable upper jaw component per se;

FIG. 11 is a diagrammatic top view taken in the direction of arrow 11 in FIG. 10;

FIG. 12 is a diagrammatic front view taken in the direction of arrow 12 in FIG. 10;

FIG. 13 is a diagrammatic top, left, front perspective view similar to FIG. 1, showing a hand of a user placed between the two pneumatic bladders before inflation of the pneumatic bladders;

FIG. 14 is a diagrammatic perspective view similar to FIG. 13, showing the movable upper jaw component lowered and the pneumatic bladders appropriately inflated to cause pressure to be applied to the hand of the user;

FIG. 15 is a diagrammatic longitudinal partial cross sectional view taken on line 15-15 in FIG. 1, when the pneumatic bladders are deflated;

FIG. 16 is a diagrammatic longitudinal partial cross sectional view taken on line 16-16 in FIG. 1, when the pneumatic bladders are inflated;

FIG. 17 is a block diagram showing the interconnection of the circuitry and components of the present invention; and

FIG. 18 is a timing diagram showing the relationship of the sequence of events occurring among the various components and circuitry of the present invention during the operative cycle thereof.

### A MARSHALING OF REFERENCE NUMERALS UTILIZED IN THE DRAWING

20 device
22 jaw assembly
24 front opening
26 first pneumatic bladder
30 second pneumatic bladder
34 enclosure
36 stationary lower jaw component
38 movable upper jaw component
40 mechanisms for inflating and deflating at least one pneumatic bladder (26, 30)
41 mechanisms for fixedly adjusting jaws
42 DC power source
44 voltage regulator
46 on/off power switch
48 control circuit
50 pump
53 air outlet, of said pump 50
52 air inlet valve
54 normally opened solenoid valve
56 air vent of normally opened solenoid valve 54
57 air inlet of normally opened solenoid valve 54
58 pressure sensor
60 on interval timer
62 off interval timer
64 manually adjustable pressure switch
66 optional timer
68 first manual control for first timer circuit 60
70 second manual control for second timer circuit 62
74 separate distinct reference values components

### DETAILED DESCRIPTION OF THE PREFERRED EMBODIMENT

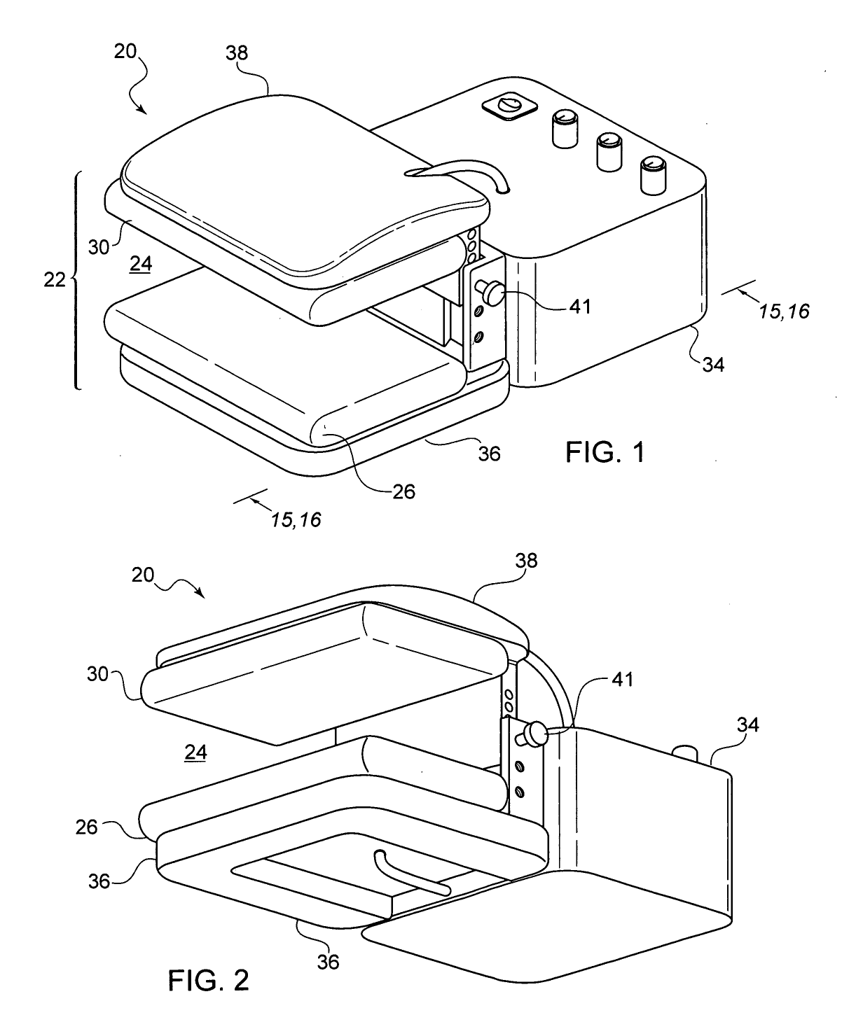

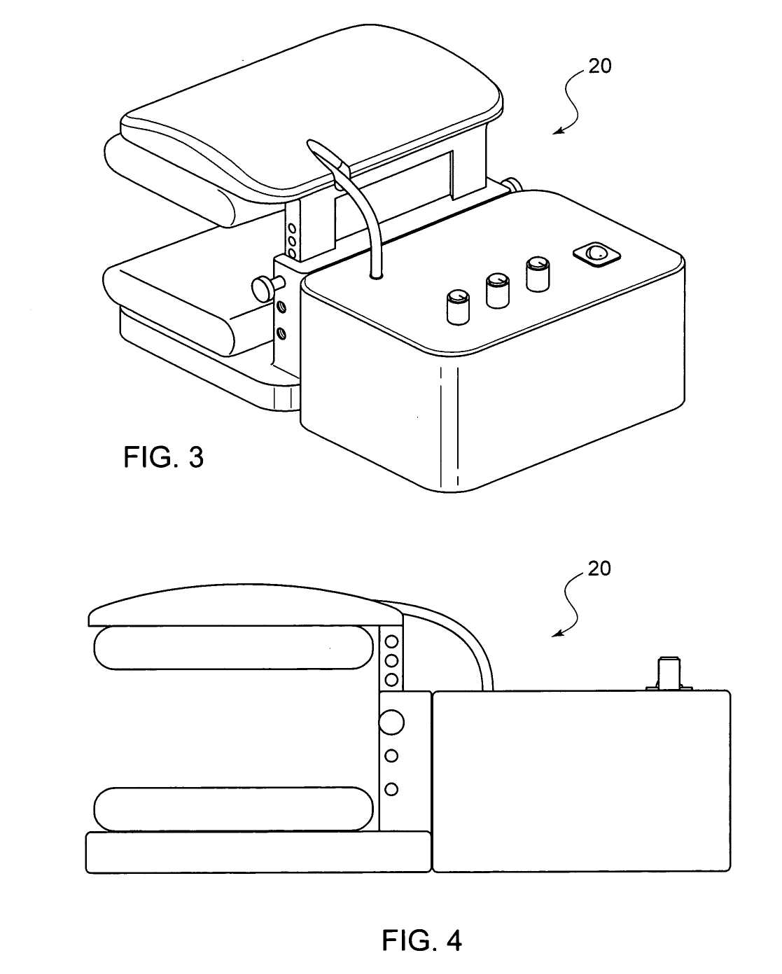

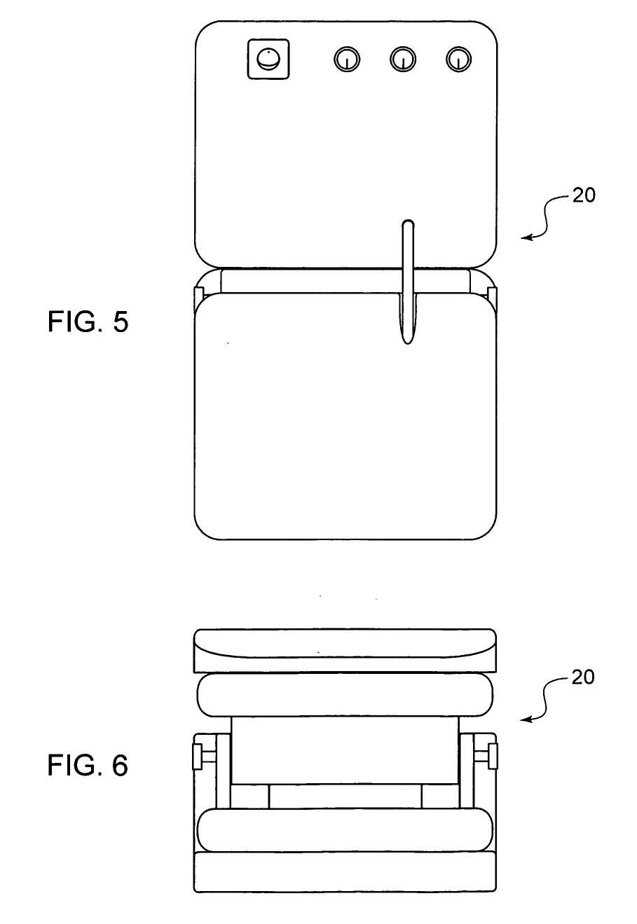

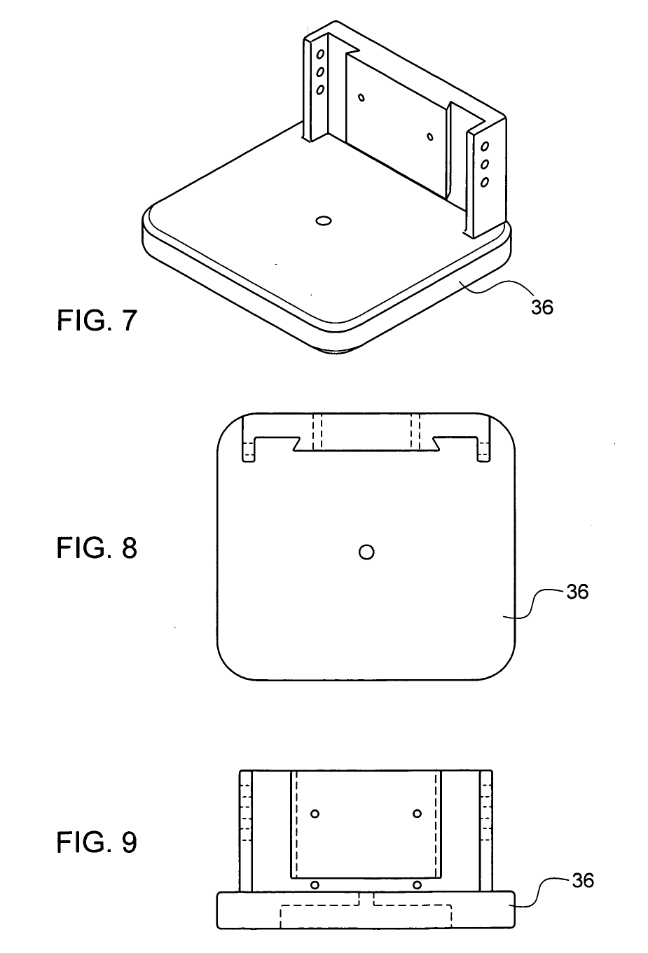

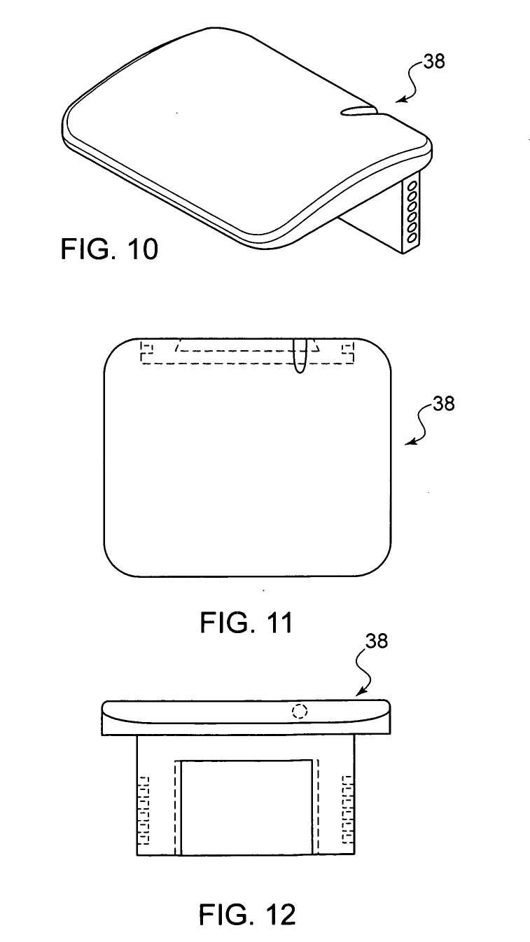

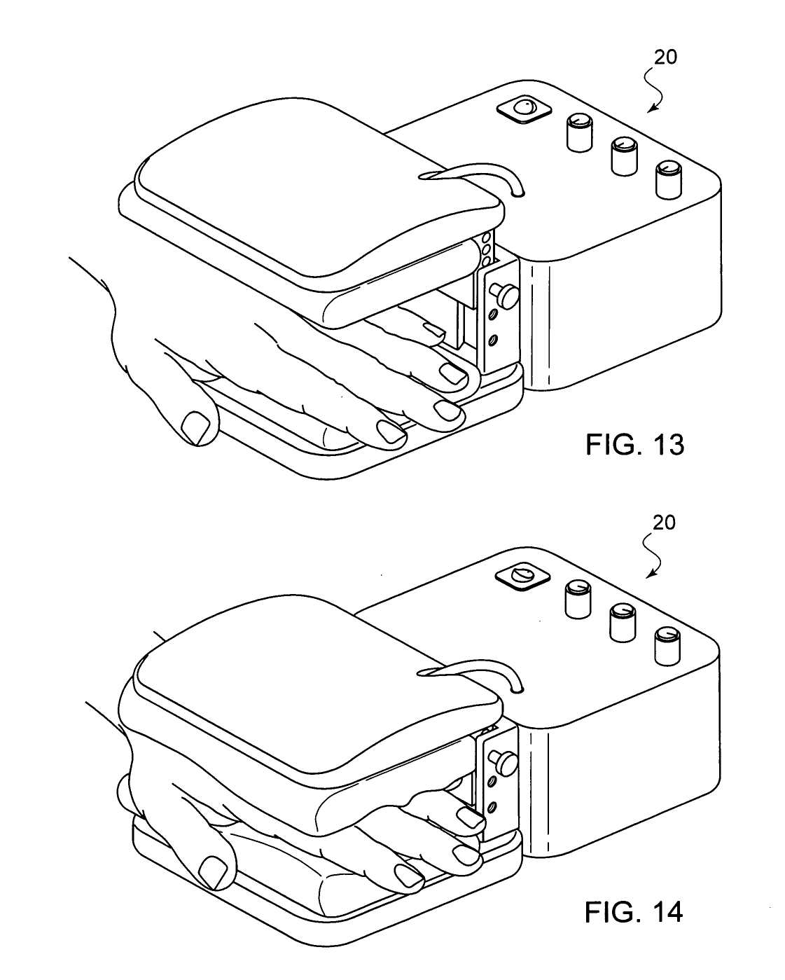

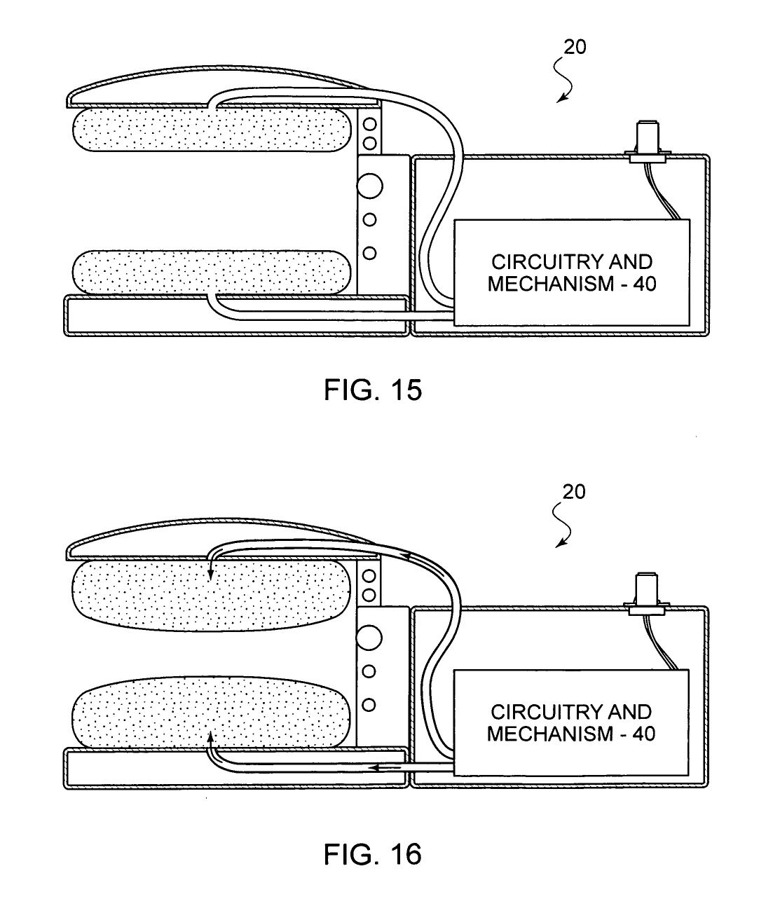

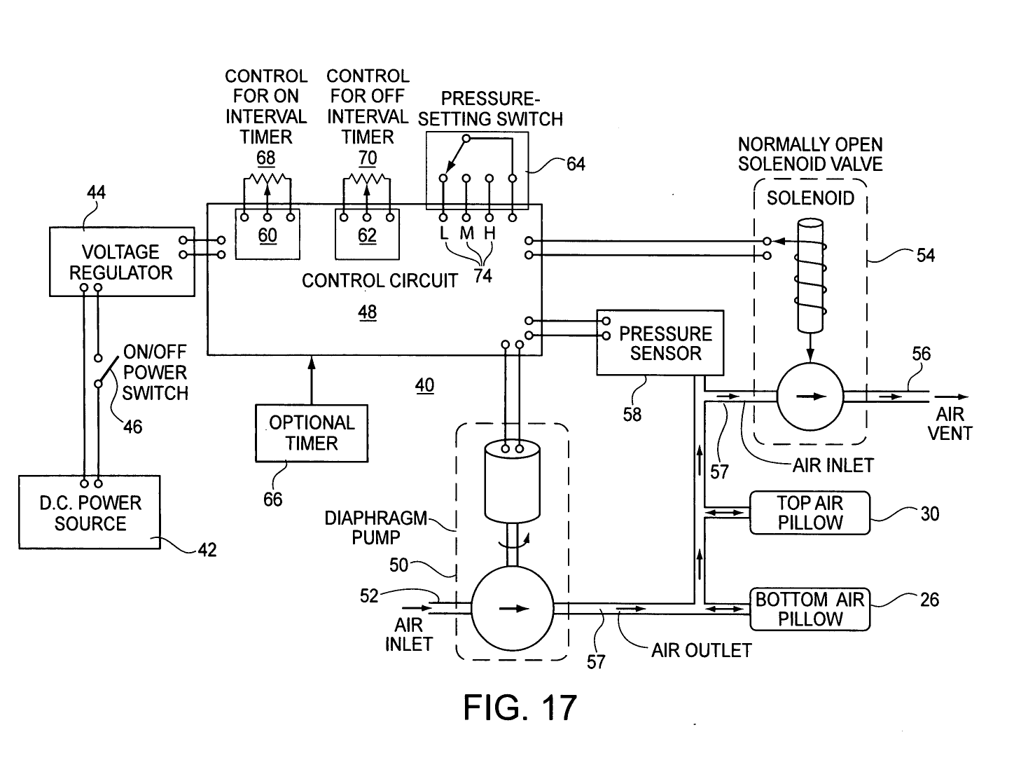

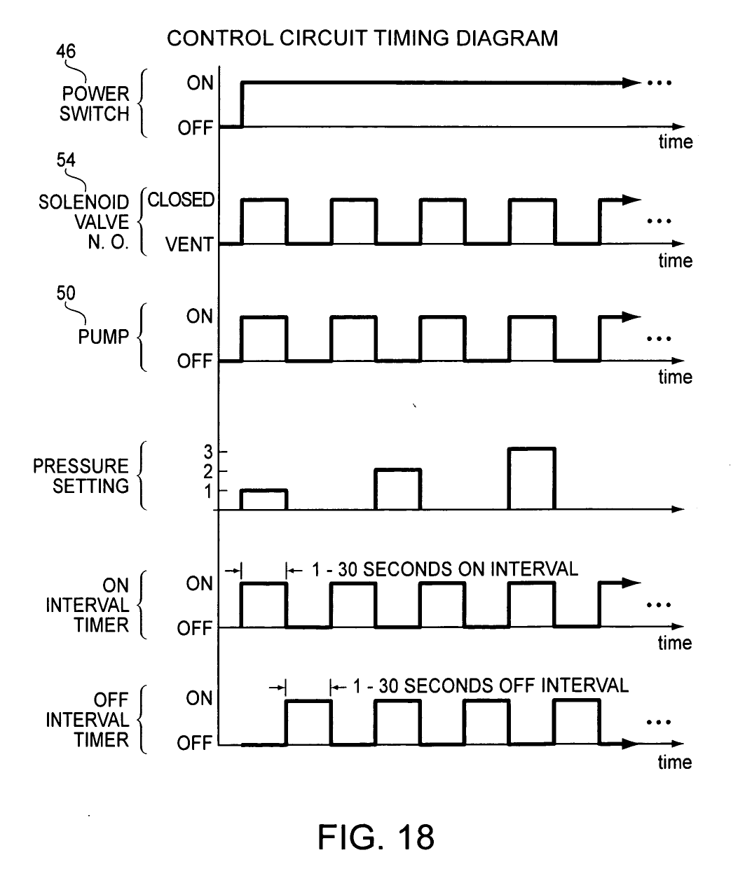

Referring now to the figures, in which like numerals indicate like parts, and particularly to FIGS. 1, through 18, which are a diagrammatic top, left, front perspective view of an embodiment of the present invention; a diagrammatic bottom, left, front perspective view thereof; a diagrammatic top, left, rear perspective view thereof; a diagrammatic left side view taken in the direction of arrow 4 in FIG. 1; a diagrammatic top view taken in the direction of arrow 5 in FIG. 1; a diagrammatic front view taken in the direction of arrow 6 in FIG. 1; a diagrammatic top, left, front perspective view of the stationary lower jaw component per se; a diagrammatic top view taken in the direction of arrow 8 in FIG. 7; a diagrammatic front view taken in the direction of arrow 9 in FIG. 7; a diagrammatic top, left, front perspective view of the movable upper jaw component per se; a diagrammatic top view taken in the direction of arrow 11 in FIG. 10; a diagrammatic front view taken in the direction of arrow 12 in FIG. 10; a diagrammatic top, left, front perspective view similar to FIG. 1, showing a hand of a user placed between the two pneumatic bladders before inflation of the pneumatic bladders; a diagrammatic view similar to FIG. 13, showing the movable upper jaw component lowered and the pneumatic bladders appropriately inflated to cause pressure to be applied to the hand of the user; a diagrammatic longitudinal partial cross sectional view taken on line 15-15 in FIG. 1, when the pneumatic bladders are deflated; a diagrammatic longitudinal partial cross sectional view taken on line 16-16 in FIG. 1, when the pneumatic bladders are inflated; a block diagram showing the interconnection of the circuitry and components of the present invention; and a timing diagram showing the relationship of the sequence of events occurring among the various components and circuitry of the present invention during the operative cycle thereof, and as such, will be discussed with reference thereto.

The present invention is a device 20 for providing therapeutic pressure therapy to parts of the body for treatment of pain which comprises the following components. First a jaw assembly 22 having a front opening 24 to receive the part of the body therein. Second, a first pneumatic bladder 26 secured to a first stationary lower jaw component 36 of said jaw assembly 22 at the bottom of said front opening 24. Third, a second pneumatic bladder 30 secured to a second movable upper jaw component 38 of said jaw assembly 22 at the top of said front opening 24 directly above said first pneumatic bladder 26. Fourth an enclosure 34 behind said jaw assembly 22. Fifth, mechanisms 40 within said enclosure 34 for inflating and deflating at least one of said first pneumatic bladder 26 and said second pneumatic bladder 30.

The invention comprises the device as described initially, with the addition that said jaw assembly 22 comprises: a stationary lower jaw component 36 a movable upper jaw component 38, and mechanisms 41 for fixedly adjusting said movable upper jaw component 38 closer and further with respect to said stationary lower jaw component 36. Said mechanisms 41 may, in the preferred embodiment of the present invention may utilize an indexing mechanism to allow said movable upper jaw component 38 to be held at a plurality of positions relative to said stationary lower jaw component 36.

The invention comprises the device as described initially, with the addition that said inflating and deflating mechanisms has the following parts. First a DC power source 42. Second a voltage regulator 44 electrically connected to said DC power source 42. Third an on/off power switch 46 electrically connected between said DC power source 42 and said voltage regulator 44. Fourth a control circuit 48 electrically connected to said voltage regulator 44. Fifth a pump 50 having an air inlet 52 and an air outlet 53, said pump 50 electrically connected to said control circuit 48 while said air outlet 53 is fluidly connected to said pneumatic bladders 26, 30. Sixth a normally opened solenoid valve 54 having an air vent 56, said solenoid valve 54 electrically connected to said control circuit 48. Seventh, and a pressure sensor 58 electrically connected to said control circuit 48 and fluidly connected to said air inlet 53 of said pump 50, said air inlet 57 of said solenoid valve 54, and said pneumatic bladders 26, 30.

The invention comprises the device as described initially including the inflating and deflating mechanisms 40 as described wherein said control circuit 48 comprises: an on interval timer 60 having a control 68; an off interval timer 62 having a control 70; and a manually adjustable pressure switch 64 for choosing a reference element 74.

The invention comprises the device as described further comprising an optional timer 66 electrically connected to said control circuit 48, for disconnecting the electrical power source 42 and accordingly deactivating and shutting off said device 20.

The invention comprises the device as described initially wherein said deflating and inflating mechanisms 40 comprises: an electrically operated pump 50; an electrically operated solenoid valve 54; a pressure sensor component 58; a first manual control 68 for a first timer circuit 60 and a second manual control 70 for a second timer circuit 62; a first timer circuit 60, a second timer circuit 62; and a manually adjustable switch 64 for choosing a reference element 74 and feedback control circuitry in communication with said electrically operated pump 50, said pressure sensor component 58 and said reference value elements 74, wherein a pressure in both said first pneumatic bladder 26 and said second pneumatic bladder 30 are responsive to the value chosen with said manually adjustable pressure switch 64 for choosing a reference element 74 typically L, M, or H.

The invention comprises the device as described, wherein said electrically operated pump 50 is a diaphragm pump.

The invention comprises the device as described wherein said manually adjustable switch 64 for choosing a reference element 74 comprises a manually adjustable pressure switch 64 having at least two positions for selecting at least two corresponding separate distinct value reference components 74, connected thereto, which are utilized as reference elements.

An embodiment of the invention comprises the device as described wherein said distinct value reference components 74 are resistors.

An alternate embodiment of the invention comprises the device as described wherein said distinct value reference components 74 are zener diodes.

An alternate embodiment of the invention comprises the device as described wherein said distinct value reference components 74 are the series combination of a zener diode and a resistor.

The invention comprises the device as described wherein said manually adjustable reference element 74 comprises a variable potentiometer having an infinite number of positions for selecting a resistive value, which is utilized as a reference element.

The invention comprises the device as described wherein said manually chosen reference element 74 comprises a variably digital component having a number of positions for selecting a digital output values which is utilized as a reference element.

The invention comprises the device as described wherein said deflating and inflating mechanisms 40 further comprises: said first timer circuit 60 which controls the length of the ON interval period during which said electrically operated pump is enabled; and said second timer circuit 62 which controls the length of the OFF interval period during which said electrically operated pump is disabled.

The invention comprises the device as described wherein said deflating and inflating mechanisms 40 further comprises: said first timer circuit 60 wherein length of the ON interval period during which said electrically operated pump 50 is enabled is responsive to a position set on a first manual adjustable control component 68.

The invention comprises the device as described wherein said deflating and inflating mechanisms 40 further comprises: a second timer circuit 62 wherein length of the OFF interval period during which said electrically operated pump 50 is disabled is responsive to a position set on a second manual adjustable control component 70.

The invention comprises the device as described wherein said deflating and inflating mechanisms 40 further comprises: said first timer circuit 60 which controls the length of the ON interval period during which said electrically operated solenoid valve 54 is closed; and said second timer circuit 62 which controls the length of the OFF interval period during which said electrically operated solenoid valve 54 is open.

The invention comprises the device as described wherein said deflating and inflating mechanisms 40 further comprises: said first timer circuit 60 wherein length of the ON interval period during which said electrically operated solenoid valve 54 is closed is responsive to a position set on said first manual adjustable control component 68.

The invention comprises the device as described wherein said deflating and inflating mechanisms 40 further comprises: said second timer circuit 62 wherein length of the OFF interval period during which said electrically operated solenoid valve 54 is open is responsive to a position set on a second manual adjustable control component 70.

The invention comprises the device as described wherein said first manual control 68 and said second manual control 70 may each individually be selected from the group consisting of: a potentiometer, a selector switch, and a digital component having a number of positions for selecting a digital output value.

While the invention has been illustrated and described as embodiments of a device for providing therapeutic pressure therapy to parts of the body for treatment of pain, accordingly it is not limited to the details shown, since it will be understood that various omissions, modifications, substitutions and changes in the forms and details of the device illustrated and its operation can be made by those skilled in the art without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute characteristics of the generic or specific aspects of this invention.

The invention claimed is:

1. A device for providing therapeutic pressure therapy to parts of the body for treatment of pain which comprises:
a) a jaw assembly having a front opening to receive the part of the body therein;
b) a first pneumatic bladder secured to a first jaw of said jaw assembly at the bottom of said front opening;
c) a second pneumatic bladder secured to a second jaw of said jaw assembly at the top of said front opening directly above said first pneumatic bladder;
d) an enclosure behind said jaw assembly; and
e) means within said enclosure for inflating and deflating said first pneumatic bladder and said second pneumatic bladder.

2. The device as recited in claim 1, wherein said jaw assembly comprises:
a) a stationary lower jaw component;
b) a movable upper jaw component; and
c) means for fixedly adjusting said movable upper jaw component closer and further with respect to said stationary lower jaw component.

3. The device as recited in claim 1, wherein said inflating and deflating means comprises:
a) a DC power source;
b) a voltage regulator electrically connected to said DC power source;
c) an on/off power switch electrically connected between said DC power source and said voltage regulator;
d) a control circuit electrically connected to said voltage regulator;
e) a pump operating an air inlet valve, said pump electrically connected to said control circuit while said air valve is fluidly connected to said pneumatic bladders;
f) a normally opened solenoid valve having an air vent, said solenoid valve electrically connected to said control circuit; and
g) a pressure sensor electrically connected to said control circuit and fluidly connected between said air inlet valve of said pump and said solenoid valve.

4. The device as recited in claim 3, wherein said control circuit comprises:
a) an on interval timer having a control means;
b) an off interval timer having a control means; and
c) a pressure setting switch.

5. The device as recited in claim 3, further comprising an optional timer electrically connected to said control circuit, for disconnecting the electrical power source and accordingly deactivating and shutting off said device.

6. The device as recited in claim 1, wherein said inflating and deflating means comprises:
a) an electrically operated pump;
b) an electrically operated solenoid valve;
c) a pressure sensor component;
d) a first manual control and a second manual control;
e) a first timer circuit, a second timer circuit; and
f) a manually adjustable reference element and feedback control circuitry in communication with said electrically operated pump, said pressure sensor component and said reference value element, wherein a pressure in both said first pneumatic bladder and said second pneumatic bladder are responsive to the value chosen for said manually adjustable reference element.

7. The device as recited in claim 6, wherein said electrically operated pump is a diaphragm pump.

8. The device as recited in claim 6, wherein said manually adjustable reference element comprises a selector switch having at least two positions for selecting at least two corresponding separate distinct values components, connected thereto, which are utilized as reference elements.

9. The device as recited in claim 6, wherein said distinct values components are resistors.

10. The device as recited in claim 6, wherein said distinct values components are zener diodes.

11. The device as recited in claim 6, wherein said distinct values components are the series combination of a zener diode and a resistor.

12. The device as recited in claim 6, wherein said manually adjustable reference element comprises a variable potentiometer having an infinite number of positions for selecting a resistive value, which is utilized as reference elements.

13. The device as recited in claim 6, wherein said manually adjustable reference element comprises a variably digital component having a number of positions for selecting a digital output values which is utilized as reference elements.

14. The device as recited in claim 6, wherein said inflating means further comprises:
a) said first timer circuit which controls the length of the ON interval period during which said electrically operated pump is enabled; and
b) said second timer circuit which controls the length of the OFF interval period during which said electrically operated pump is disabled.

15. The device as recited in claim 14, wherein said inflating means further comprises: said first timer circuit wherein length of the ON interval period during which said electrically operated pump is enabled is responsive to a position set on a first manual adjustable control component.

16. The device as recited in claim 14, wherein said deflating means further comprises: a second timer circuit wherein length of the OFF interval period during which said electrically operated pump is disabled is responsive to a position set on a second manual adjustable control component.

17. The device as recited in claim 6, wherein said deflating means further comprises:
a) said first timer circuit which controls the length of the ON interval period during which said electrically operated solenoid valve is closed; and
b) said second timer circuit which controls the length of the OFF interval period during which said electrically operated solenoid valve is open.

18. The device as recited in claim 17, wherein said deflating means further comprises: said first timer circuit wherein length of the ON interval period during which said electrically solenoid valve is closed is responsive to a position set on said first manual adjustable control component.

19. The device as recited in claim 17, wherein said deflating means further comprises: said second timer circuit wherein length of the OFF interval period during which said electrically operated solenoid valve is open is responsive to a position set on a second manual adjustable control component.

20. The device as recited in claim 6, wherein said first manual control and said second manual control may each individually be selected from the group consisting of:
a) a potentiometer and
b) a selector switch and
c) a digital component having a number of positions for selecting a digital output values.
